(19) United States
(12) Patent Application Publication
(10) Pub. No.: US 2007/0149590 A1
(43) Pub. Date: Jun. 28, 2007

(54) Controlled release dosage form of pirazole compounds

(76) Inventor: Francisco Javier Lizcano Garcia, Barcelona (ES)

Correspondence Address: COOPER & DUNHAM, LLP, 1185 AVENUE OF THE AMERICAS, NEW YORK, NY, 10036, US

(21) Appl. No.: 11/498,213

(22) Filed: Aug. 1, 2006

Related U.S. Application Data
(60) Provisional application No. 60/705,840, filed on Aug. 5, 2005.

(51) Int. Cl. A61K 31/416

(52) U.S. Cl. 514/406

(57) ABSTRACT

The present invention refers to a pharmaceutical composition for oral administration of pirazole compounds.

### FIELD OF THE INVENTION

The present invention refers to a pharmaceutical composition for oral administration of pirazole compounds.

### BACKGROUND OF THE INVENTION

Pirazole compounds described in general formula XI and especially X (with R₁ being either H or Methyl)) are known as pharmaceutically active compounds.

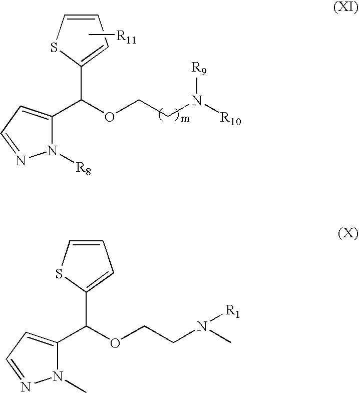

Especially 2-((1-methyl-1H-pyrazol-5-yl)(thiophen-2-yl)methoxy)-N,N-dimethylethanamine of the formula (I) 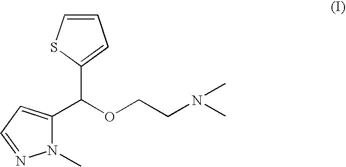
is already described in U.S. Pat. No. 6,410,582 and EP 1 072 266 including its synthesis and analgesic properties, whereas US 2003/022925, US 2004/0142929 and EP 1 413 305 A1 claim its use for urinary incontinence, also disclosing some pharmaceutical compositions for the oral administration of 2-((1-methyl-1H-pyrazol-5-yl)(thiophen-2-yl)methoxy)-N,N-dimethylethanamine citrate. Furthermore EP 1 086 682 B1 describes the use of cizoliitine for the treatment of neurogenic inflammation, U.S. Pat. No. 6,518,295 B1 and EP 1 103 243 B1 the use for the treatment of disorders mediated by excess of substance P and EP 1 384 476 A1 and US2004/0142994 the use for the treatment of respiratory diseases.

Another aspect of the invention relates to the pure enantiomers of 2-((1-methyl-1H-pyrazol-5-yl)(thiophen-2-yl)methoxy)-N,N-dimethylethanamine (formulas (II) and (III)).

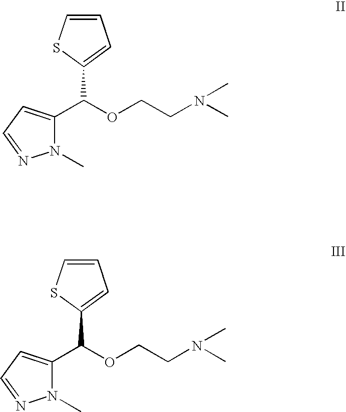

2-((1-methyl-1H-pyrazol-5-yl)(thiophen-2-yl)methoxy)-N-methylethanamine of formula (IV) is known as a metabolite of 2-((1-methyl-1H-pyrazol-5-yl) (thiophen-2-yl)methoxy)-N,N-dimethylethanamine and also its enantiomers (formulas (V) and (VI)) are of high interest in regards of this invention.

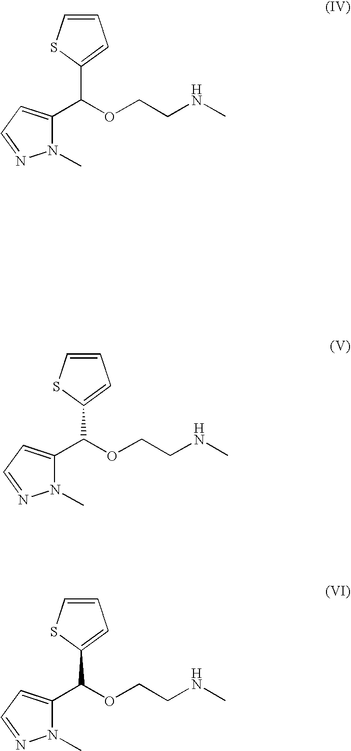

All of the cited patents/publications are included here by reference.

### SUMMARY OF THE INVENTION

This application provides a pharmaceutical composition for the oral administration of a pirazole compound of formula (XI) 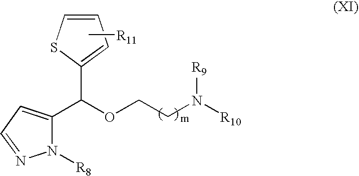
with
m is 1 or 2;
R₈ is selected from hydrogen, fluoride, chloride, bromide and C₁₋₄-Alkyl, especially methyl;
R₉ and R₁₀ are independently selected from C₍₁₋₄₎-Alkyl or together with the Nitrogen form an azaheterocyclic ring;
R₁₁ is selected from the group consisting of hydrogen, fluoride, chloride, bromide, C₁₋₄-Alkyl, especially methyl or trifluoromethyl, and O—C₁₋₄-Alkyl, especially methoxy,
of one of its enantiomers or a mixture of its enantiomers in any mixing ratio including the racemate,
including pharmaceutically acceptable salts or solvates, (collectively called active principle hereafter)
comprising:
≧70% by weight of the active principle in at least one of its distinct parts.

This application also provides a pharmaceutical composition according to claim 1 for the oral administration of (+)-2-((1-methyl-1H-pyrazol-5-yl)(thiophen-2-yl)methoxy)-N,N-dimethylethanamine or (−)-2-((1-methyl-1H-pyrazol-5-yl)(thiophen-2-yl)methoxy)-N,N-dimethylethanamine according to formula II or III respectively or any mixture of the compounds of formula II or III including a racemic mixture (collectively called active principle hereafter) 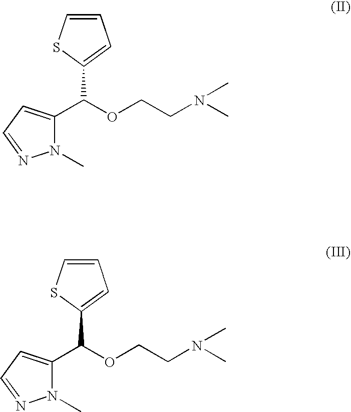
including pharmaceutically acceptable salts or solvates,
comprising:
≧70% by weight of the active principle
in at least one of its distinct parts.

This application also provides a pharmaceutical composition according to claim 1 for the oral administration of (+)-2-((1-methyl-1H-pyrazol-5-yl) (thiophen-2-yl)methoxy)-N-methylethanamine or (−)-2-((1-methyl-1H-pyrazol-5-yl)(thiophen-2-yl)methoxy)-N-methylethanamine according to formula V or VI respectively or any mixture of the compounds of formula V or VI including a racemic mixture (collectively called active principle hereafter) 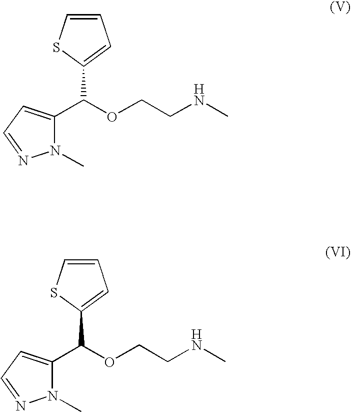
including pharmaceutically acceptable salts or solvates,
comprising:
≧70% by weight of the active principle
in at least one of its distinct parts.

This application further provides a pharmaceutical composition for the oral administration of a pirazole compound of formula (XI) 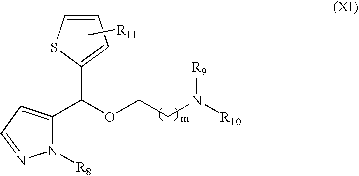
with
m is 1 or 2;
R₈ is selected from hydrogen, fluoride, chloride, bromide and C₁₋₄-Alkyl, especially methyl;
R₉ and R₁₀ are independently selected from C₍₁₋₄₎-Alkyl or together with the Nitrogen form an azaheterocyclic ring;
R₁₁ is selected from the group consisting of hydrogen, fluoride, chloride, bromide, C₁₋₄-Alkyl, especially methyl or trifluoromethyl, and O—C₁₋₄-Alkyl, especially methoxy,
of one of its enantiomers or a mixture of its enantiomers in any mixing ratio including the racemate,
including pharmaceutically acceptable salts or solvates, (collectively called active principle hereafter)
comprising:
(a) a core comprising the active principle and a Extrusion/Spheronisation enhancer,
(b) a coating comprising a coating agent.

This application provides a Pharmaceutical composition according to claim 20 for the oral administration of (+)-2-((1-methyl-1H-pyrazol-5-yl)(thiophen-2-yl)methoxy)-N,N-dimethylethanamine or (−)-2-((1-methyl-1H-pyrazol-5-yl)(thiophen-2-yl)methoxy)-N,N-dimethylethanamine according to formula II or III respectively or any mixture of the compounds of formula II or III including a racemic mixture (collectively called active principle hereafter) 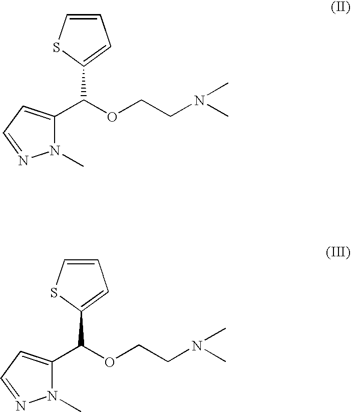
including pharmaceutically acceptable salts or solvates, comprising:
(a) a core comprising the active principle and a Extrusion/Spheronisation enhancer,
(b) a coating comprising a coating agent.

This application also provides a pharmaceutical composition according to claim 4 for the oral administration of (+)-2-((1-methyl-1H-pyrazol-5-yl)(thiophen-2-yl)methoxy)-N-methylethanamine or (−)-2-((1-methyl-1H-pyrazol-5-yl)(thiophen-2-yl)methoxy)-N-methylethanamine according to formula V or VI respectively or any mixture of the compounds of formula V or VI including a racemic mixture (collectively called active principle hereafter) 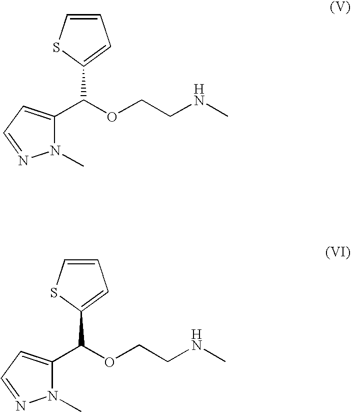
including pharmaceutically acceptable salts or solvates,
comprising:
(a) a core comprising the active principle and a Extrusion/Spheronisation enhancer,
(b) a coating comprising a coating agent.

### DETAILED DESCRIPTION OF THE INVENTION

The present invention refers to a pharmaceutical composition for oral administration of pirazole compounds, especially 2-((1-methyl-1H-pyrazol-5-yl)(thiophen-2-yl)methoxy)-N,N-dimethylethanamine, its metabolites and/or its enantiomers, and their physiologically acceptable salts and solvates.

The oral pharmaceutical compositions already described for 2-((1-methyl-1H-pyrazol-5-yl)(thiophen-2-yl)methoxy)-N,N-dimethylethanamine in the state of the art are more designed to allow the application of moderate amounts of the active compound/principle (often in fairly high volumes of the composition), which might not be sufficient for all suitable treatments. Thus there was a medical need for alternative oral pharmaceutical compositions. The oral pharmaceutical compositions according to this invention allow for the application of larger amounts of the compound and especially more compound per unit of volume of the pharmaceutical composition. As a consequence an oral once-daily pharmaceutical composition preferably with a slow release of the pirazole compounds, especially 2-((1-methyl-1H-pyrazol-5-yl)(thiophen-2-yl)methoxy)-N,N-dimethylethanamine, became available.

The pharmaceutical compositions of the present invention are especially suitable for the treatment of urinary incontinence, including imperative micturition or urge incontinence, hyperreflexia, urinary stress incontinence, mixed incontinence and enuresis, as well as e.g as analgesics and the other indications described especially for 2-((1-methyl-1H-pyrazol-5-yl)(thiophen-2-yl)methoxy)-N,N-dimethylethanamine its metabolite and/or its enantiomers.

Urination is a function of the lower urinary tract that is defined as discharge of urine through the urethra. Urination is considered to be normal in an adult when it is voluntary, continuous, complete, satisfactory, interruptible, spaced out in time (at socially acceptable intervals), without causing abdominal pressure, without urgency, and only occasional at night.

Urinary incontinence, a urinary disorder, is defined as the involuntarily discharge of urine, which can be demonstrated objectively. This functional disorder of bladder is a health problem of increasing social and hygienic relevance for the population that suffers from it. According to our data, urinary incontinence occurs in approximately 1.5 to 5% of men and 10 to 30% of women in the population between 15 and 64 years old. However, if we select the non-hospitalised population sector over 60 years old, the prevalence ranges from 15% to 35% of this population. On the other hand, when hospitalised patients over 60 years old are studied, the incidence is higher. Urinary incontinence affects approximately 2 million of the Spanish population.

Urinary incontinence can be considered as a symptom, sign or pathological condition. The following is one of the possible classifications of this functional disorder.

Imperative micturition or urge incontinence. This is when the involuntary discharge of urine is accompanied by an intense desire to urinate (urgency). This can be separated into motor urgency incontinence or sensitive urgency incontinence. Motor urgency incontinence is associated with hyperactivity of the detrusor muscle and/or reduced distensibility of the detrusor. Hyperactivity is characterised by involuntary contractions of the detrusor during the filling stage, either spontaneous or provoked, that the patient cannot totally suppress. Hyperactivity of the detrusor muscle can occur when there is obstruction of the exiting urinary flow, inflammation and conditions in which the bladder is irritated, or it can be of unknown aetiology (idiopathic).

Hyperreflexia, is described as a condition that presents uncontrolled contractions of the detrusor muscle associated with neurological disorders such as multiple sclerosis or plaque sclerosis, sequelae of medular traumatisms or Parkinson's disease.

Urinary stress incontinence, due to a defective urethral closure mechanism, there is involuntary discharge of urine in the absence of detrusor contraction that occurs when the intravesical pressure exceeds the pressure in the urethra. Involuntary discharge occurs when some physical exertion is made such as jumping, coughing, going down stairs etc. One additional factor can be due to structural changes in the urethra due to menopausal hypooestrogenia.

Mixed incontinence, this term refers to the existence of both urgency incontinence and stress incontinence.

Enuresis, this term refers to any involuntary loss of urine and more specifically refers to incontinence during sleep. It most often applies to children with a higher incidence in boys and in the age group of up to 5 years.

For more definitions and a standardisation of Terminology reference is made to Abrams et al, Neurology and Urodynamics 21:167-178 (2002)

“Distinct parts” as used in this application is intended to be any part of the pharmaceutical formulation which has either physically or by the non-continuous distribution of chemistry a distinct area. An example would be the core of the pellet or the core of the pellet core.

The term “lower C₍₁₋₄₎-Alkyl”) represents a linear or branched alkyl chain radical derived from a saturated hydrocarbon of 1 to 4 carbon atoms, such as methyl, ethyl, propyl, isopropyl, butyl, isobutyl, sec-butyl and tert-butyl.

The term “O—C₍₁₋₄₎-Alkyl”) represents a linear or branched alkyl chain radical derived from a saturated hydrocarbon of 1 to 4 carbon atoms, such as methyl, ethyl, propyl, isopropyl, butyl, isobutyl, sec-butyl and tert-butyl, connected to the core molecule by an ether bond.

The term “salt” is to be understood as meaning any form of the active compound according to the invention in which this assumes an ionic form or is charged and is coupled with a counter-ion (a cation or anion) or is in solution. By this are also to be understood complexes of the active compound with other molecules and ions, in particular complexes which are complexed via ionic interactions.

The term “pharmaceutically acceptable salt” is understood in particular, in the context of this invention, as salt formed either with a) a pharmaceutically acceptable acid or b) pharmaceutically acceptable base. Especially this means salts of the particular active compound with inorganic or organic acids which are pharmaceutically acceptable—especially if used on humans and/or mammals—or with at least one, preferably inorganic, cation that is pharmaceutically acceptable—especially if used on humans and/or mammals. Examples of pharmaceutically acceptable salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1,2-dihydrolb6-benzo[d]isothiazol-3-one (saccharin acid), monomethylsebacic acid, 5-oxo-proline, hexane-1-sulfonic acid, nicotinic acid, 2-, 3- or 4-aminobenzoic acid, 2,4,6-trimethyl-benzoic acid, alpha-lipoic acid, acetylglycine, acetylsalicylic acid, hippuric acid and/or aspartic acid. Examples of pharmaceutically acceptable salts of particular bases are salts of alkali metals and alkaline earth metals and NH₄.

In the context of this invention the preferred salt is a salt of the particular active compound with a pharmaceutically acceptable acid.

The salt particularly preferred in the context of this invention is the citrate.

The term “solvalte” is understood in particular, in the context of this invention, as a compound formed by salvation (the combination of solvent molecules with molecules or ions of the solute).

In the context of this invention the term “plasticizer” is understood as a chemical substance added to impart flexibility, workability and distensibilty. Plasticizers are used e.g. for film coating or for extrusion/spheronisation. Preferred plasticizers for this invention are macrogols, like Macrogol 8000, a polyethylene glycol. Other Plasticizers commonly used in film coating etc. and also suitable for the invention are (as examples/not limiting):
Class Polyols: Polyethylene glycols (in general i.e. PEG8000), Propylene glycol, glycerol
Class Organic esters: Diethyl phtalate, dibutyl sebacate, triethyl citrate.
Class Oil/Glycerides: Castor oil, acetylated monglycerides, fractionated coconut oil.

In the context of this invention the term “Extrusion/Spheronisation enhancer” is understood as a chemical substance added to produce a plastic mass which is easily deformed and extrudes at low pressures. Extrusion/Spheronisation enhancer are used e.g. for allowing a satisfying extrusion and/or following spheronisation. A preferred Extrusion/Spheronisation enhancer for this invention is microcrystalline cellulose. For example the inclusion of 10-50% microcrystalline cellulose in a formulation will produce a plastic mass which is easily deformed and extrudes at low pressures. For particularly high drug loadings (above 80%) colloidal grades are recommended, these consist of mycrocristalline cellulose coated with binder, sodium carboxymethylcellulose. Other Extrusion/Spheronisation enhancers commonly used and also suitable for the invention are (as examples/not limiting) lactose, starch, pregelanitised starch and calcium phosphate.

In the context of this invention the term “coating agent” is understood as a chemical substance added to a coating or as a coating to produce an effect on the surface of the pharmaceutical composition or one of its subunits. The effects desired include release control, chemical stability enhancement (e.g. gastro-intestinal coating, light exclusion), physical stability enhancement (hardening, confining/conserving the surface), or e.g. coloring. In a preferred sense “coating agent” is understood as “release control coating agent” a chemical substance added to or as a coating to control the release of the active compound/principle from the pharmaceutical composition or from one of its subunits. A preferred coating agent for this invention is ethylcellulose. In a preferred embodiment the ethylcellulose is used as a pseudo-latex containing plasticiser already incorporated into dispersed particles in water or “in situ” with ethylcellulose plus organic solvents or solvent mixture. Other coating agents, especially for release control, commonly used and also suitable for the invention are (as examples/not limiting):
Fats and waxes (i.e. glyceril monostearate, beesxaw, carnauba wax . . . ) These coatings normally function by erosion and not by diffusion as ethylcellulose.
Acrylic polimers (polymethacrilates): Eudragit series.
Aquacoat (FMC): Ethylcellulose dispersion, a pseudo-latex stabilised in water with sodium lauryl sulphate.
Another types of celluloses: HPMC (Hydroxypropilmethylcellulose), HPC (Hydroxypropilcellulose), Methylcellulose, etc.

One main aspect of the invention is a pharmaceutical composition (called hereafter pharmaceutical composition A) for the oral administration of a pirazole compound of formula (XI) 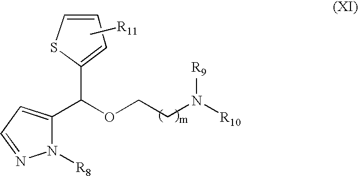
with
m is 1 or 2;
R₈ is selected from hydrogen, fluoride, chloride, bromide and C₁₋₄-Alkyl, especially methyl;
R₉ and R₁₀ are independently selected from C₍₁₋₄₎-Alkyl or together with the Nitrogen form an azaheterocyclic ring;
R₁₁ is selected from the group consisting of hydrogen, fluoride, chloride, bromide, C₁₋₄-Alkyl, especially methyl or trifluoromethyl, and O—C₁₋₄-Alkyl, especially methoxy,
of one of its enantiomers or a mixture of its enantiomers in any mixing ratio including the racemate,
including pharmaceutically acceptable salts or solvates, (collectively called active principle hereafter)
comprising:
≧70% by weight of the active principle
in at least one of its distinct parts.

A preferred embodiment of the pharmaceutical composition A is a pharmaceutical composition for the oral administration of a pirazole compound of formula (X) 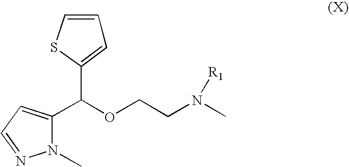
with R₁ being H or CH₃,
of one of its enantiomers or a mixture of its enantiomers in any mixing ratio including the racemate,
including pharmaceutically acceptable salts or solvates, (collectively called active principle hereafter)
comprising:
≧70% by weight of the active principle
in at least one of its distinct parts.

Another preferred embodiment of the pharmaceutical composition A is a pharmaceutical composition for the oral administration of 2-((1-methyl-1H-pyrazol-5-yl)(thiophen-2-yl)methoxy)-N,N-dimethylethanamine of formula I 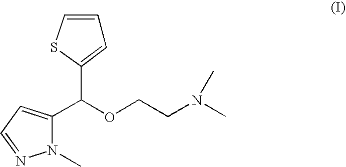
of one of its enantiomers or a mixture of its enantiomers in any mixing ratio including the racemate,
including pharmaceutically acceptable salts or solvates,
(collectively called active principle hereafter)
comprising:
≧70% by weight of the active principle
in at least one of its distinct parts.

Another preferred embodiment of the pharmaceutical composition A is a pharmaceutical composition for the oral administration of (+)-2-((1-methyl-1H-pyrazol-5-yl)(thiophen-2-yl)methoxy)-N,N-dimethylethanamine or (−)-2-((1-methyl-1H-pyrazol-5-yl)(thiophen-2-yl)methoxy)-N,N-dimethylethanamine according to formula II or III respectively or any mixture of the compounds of formula II or III including a racemic mixture (collectively called active principle hereafter) 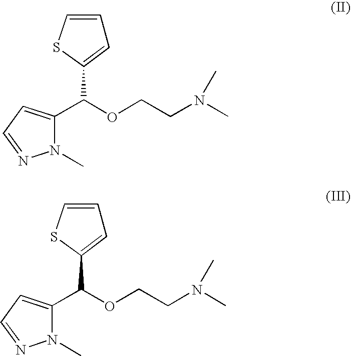
including pharmaceutically acceptable salts or solvates,
comprising:
≧70% by weight of the active principle in at least one of its distinct parts.

Another preferred embodiment of the pharmaceutical composition A is a pharmaceutical composition for the oral administration of 2-((1-methyl-1H-pyrazol-5-yl)(thiophen-2-yl)methoxy)-N-methylethanamine of formula IV 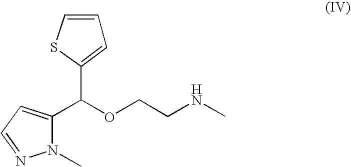
of one of its enantiomers or a mixture of its enantiomers in any mixing ratio including the racemate,
including pharmaceutically acceptable salts or solvates,
(collectively called active principle hereafter)
comprising:
≧70% by weight of the active principle
in at least one of its distinct parts.

Another preferred embodiment of the pharmaceutical composition A is a pharmaceutical composition for the oral administration of (+)-2-((1-methyl-1H-pyrazol-5-yl)(thiophen-2-yl)methoxy)-N-methylethanamine or (−)-2-((1-methyl-1H-pyrazol-5-yl)(thiophen-2-yl)methoxy)-N-methylethanamine according to formula V or VI respectively or any mixture of the compounds of formula V or VI including a racemic mixture (collectively called active principle hereafter) 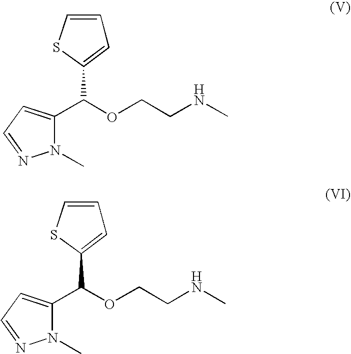
including pharmaceutically acceptable salts or solvates,
comprising:
≧70% by weight of the active principle
in at least one of its distinct parts.

In another preferred embodiment of the pharmaceutical composition A the amount of active principle is ≧500 mg and ≦1100 mg, preferably ≧600 mg and ≦1000 mg, more preferably ≧700 mg and ≦900 mg, most preferably 800 mg.

In another preferred embodiment of the pharmaceutical composition A the amount of the free base of the active principle is ≧250 mg and ≦650 mg, preferably ≧350 mg and ≦600 mg, more preferably ≧400 mg and ≦500 mg, most preferably 450 mg.

In another preferred embodiment of the pharmaceutical composition A the amount of active principle is ≧100 mg and ≦500 mg, preferably ≧150 mg and ≦450 mg, more preferably ≧200 mg and ≦400 mg.

In another preferred embodiment of the pharmaceutical composition A the amount of the free base of the active principle is ≧50 mg and ≦300 mg, preferably ≧85 mg and ≦250 mg, more preferably ≧120 mg and ≦200 mg.

In another preferred embodiment of the pharmaceutical composition A the distinct part is coated with a coating agent, preferably a release control coating agent, more preferably a cellulose derivative, most preferably ethyl cellulose.

In another preferred embodiment of the pharmaceutical composition A the distinct part comprises an extrusion/spheronisation enhancer, preferably a cellulose derivative.

In another preferred embodiment of the pharmaceutical composition A the distinct part comprises a plasticizer, preferably a polyethylene glycol, more preferably a macrogol.

In another preferred embodiment of the pharmaceutical composition A the distinct part comprises a coating agent, preferably a release control coating agent, more preferably a cellulose derivative, most preferably ethyl cellulose.

In another preferred embodiment of the pharmaceutical composition A the pharmaceutical composition is a tablet, a capsule, a pellet, a sachet or a suspension.

In another preferred embodiment of the pharmaceutical composition A the distinct part is the core of a tablet, a layer of a tablet, the nucleus of a pellet or a layer of a pellet, preferably the nucleus of a pellet.

In another preferred embodiment of the pharmaceutical composition A, the pharmaceutical composition comprises:

≧75% by weight of the active principle, preferably

≧75% to 95%, more preferably 80% to 95%,

in at least one of its distinct parts.

In another preferred embodiment of the pharmaceutical composition A, the pharmaceutical composition overall comprises:
≧60% by weight of the active principle, preferably ≧70%, preferably 70% to 95%, most preferably 75% to 95%, preferably 80% to 95%.

By “overall” is meant that the basis to calculate this percentage is the weight of the whole pharmaceutical composition.

In another preferred embodiment of the pharmaceutical composition A, the active principle is 2-((1-methyl-1H-pyrazol-5-yl)(thiophen-2-yl)methoxy)-N,N-dimethylethanamine citrate.

Another main aspect of the invention is a pharmaceutical composition (called hereafter pharmaceutical composition B) for the oral administration of a pirazole compound of formula (XI) 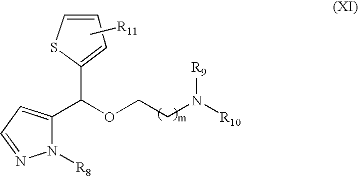
with
m is 1 or 2;
R₈ is selected from hydrogen, fluoride, chloride, bromide and C₁₋₄-Alkyl, especially methyl;
R₉ and R₁₀ are independently selected from C₍₁₋₄₎-Alkyl or together with the Nitrogen form an azaheterocyclic ring;
R₁₁ is selected from the group consisting of hydrogen, fluoride, chloride, bromide, C₁₋₄-Alkyl, especially methyl or trifluoromethyl, and O—C₁₋₄-Alkyl, especially methoxy,
of one of its enantiomers or a mixture of its enantiomers in any mixing ratio including the racemate,
including pharmaceutically acceptable salts or solvates, (collectively called active principle hereafter)
comprising:
   (a) a core comprising the active principle and a Extrusion/Spheronisation enhancer,
   (b) a coating comprising a coating agent.

Another preferred embodiment of the pharmaceutical composition B is a pharmaceutical composition for the oral administration of a pirazole compound of formula (X) 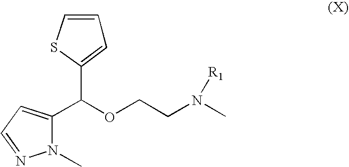
with R₁ being H or CH₃,
of one of its enantiomers or a mixture of its enantiomers in any mixing ratio including the racemate,
including pharmaceutically acceptable salts or solvates, (collectively called active principle hereafter)
comprising:
   (a) a core comprising the active principle and a Extrusion/Spheronisation enhancer,
   (b) a coating comprising a coating agent.

Another preferred embodiment of the pharmaceutical composition B is a pharmaceutical composition for the oral administration of 2-((1-methyl-1H-pyrazol-5-yl)(thiophen-2-yl)methoxy)-N,N-dimethylethanamine of formula I 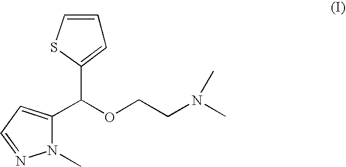
of one of its enantiomers or a mixture of its enantiomers in any mixing ratio including the racemate,
including pharmaceutically acceptable salts or solvates,
(collectively called active principle hereafter)
comprising:
   (a) a core comprising the active principle and a Extrusion/Spheronisation enhancer,
   (b) a coating comprising a coating agent.

Another preferred embodiment of the pharmaceutical composition B is a pharmaceutical composition for the oral administration of (+)-2-((1-methyl-1H-pyrazol-5-yl)(thiophen-2-yl)methoxy)-N,N-dimethylethanamine or (−)-2-((1-methyl-1H-pyrazol-5-yl)(thiophen-2-yl)methoxy)-N,N-dimethylethanamine according to formula II or III respectively or any mixture of the compounds of formula II or III including a racemic mixture (collectively called active principle hereafter) 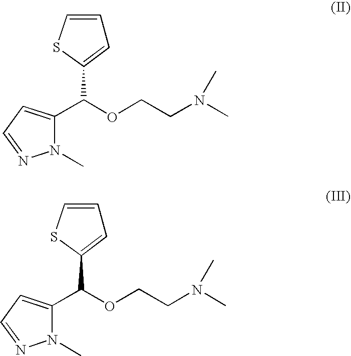
including pharmaceutically acceptable salts or solvates,
comprising:
   (a) a core comprising the active principle and a Extrusion/Spheronisation enhancer,
   (b) a coating comprising a coating agent.

Another preferred embodiment of the pharmaceutical composition B is a pharmaceutical composition for the oral administration of 2-((1-methyl-1H-pyrazol-5-yl)(thiophen-2-yl)methoxy)-N-methylethanamine of formula IV 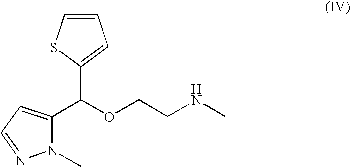
of one of its enantiomers or a mixture of its enantiomers in any mixing ratio including the racemate,
including pharmaceutically acceptable salts or solvates,
(collectively called active principle hereafter)
comprising:
   (a) a core comprising the active principle and a Extrusion/Spheronisation enhancer,
   (b) a coating comprising a coating agent.

Another preferred embodiment of the pharmaceutical composition B is a pharmaceutical composition for the oral administration of (+)-2-((1-methyl-1H-pyrazol-5-yl)(thiophen-2-yl)methoxy)-N-methylethanamine or (−)-2-((1-methyl-1H-pyrazol-5-yl)(thiophen-2-yl)methoxy)-N-methylethanamine according to formula V or VI respectively or any mixture of the compounds of formula V or VI including a racemic mixture (collectively called active principle hereafter) 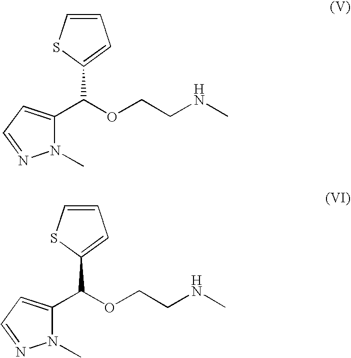
including pharmaceutically acceptable salts or solvates,
comprising:
   (a) a core comprising the active principle and a Extrusion/Spheronisation enhancer,
   (b) a coating comprising a coating agent.

In another preferred embodiment of the pharmaceutical composition B the amount of active principle is ≧500 mg and ≦1100 mg, preferably ≧600 mg and ≦1000 mg, more preferably ≧700 mg and ≦900 mg, most preferably 800 mg.

In another preferred embodiment of the pharmaceutical composition B the amount of the free base of the active principle is ≧250 mg and ≦650 mg, preferably ≧350 mg and ≦600 mg, more preferably ≧400 mg and ≦500 mg, most preferably 450 mg.

In another preferred embodiment of the pharmaceutical composition B the amount of active principle is ≧100 mg and ≦500 mg, preferably ≧150 mg and ≦450 mg, more preferably ≧200 mg and ≦400 mg.

In another preferred embodiment of the pharmaceutical composition B the amount of the free base of the active principle is ≧50 mg and ≦300 mg, preferably ≧85 mg and ≦250 mg, more preferably ≧120 mg and ≦200 mg.

In another preferred embodiment of the pharmaceutical composition B the coating agent is a release control coating agent, preferably a cellulose derivative, most preferably ethyl cellulose.

In another preferred embodiment of the pharmaceutical composition B the distinct part comprises an extrusion/spheronisation enhancer, preferably a cellulose derivative.

In another preferred embodiment of the pharmaceutical composition B the core comprises a plasticizer, preferably a polyethylene glycol, more preferably a macrogol.

In another preferred embodiment of the pharmaceutical composition B the core comprises a coating agent, preferably a release control coating agent, more preferably a cellulose derivative, most preferably ethyl cellulose.

In another preferred embodiment of the pharmaceutical composition B the Pharmaceutical Composition is a tablet, a capsule, a pellet, a sachet or a suspension.

In another preferred embodiment of the pharmaceutical composition B the core comprises:
≧65% by weight of the active principle, preferably ≧70%, more preferably 70% to 95%, most preferably 75% to 95%.

In another preferred embodiment of the pharmaceutical composition B the pharmaceutical composition overall comprises:
≧60% by weight of the active principle, preferably ≧70%, more preferably 70% to 95%, most preferably 75% to 95%.

In another preferred embodiment of the pharmaceutical composition B there is at least one additional layer between the core (a) and the coating (b), comprising an active principle and/or an ingredient.

In another preferred embodiment of the pharmaceutical composition B there is an outer layer disposed on the coating (b) comprising an active principle and/or an ingredient.

In another preferred embodiment of the pharmaceutical composition B the active principle is 2-((1-methyl-1H-pyrazol-5-yl)(thiophen-2-yl)methoxy)-N,N-dimethylethanamine citrate:

Another aspect of the invention is a process for producing a Pharmaceutical Composition A and B, which comprises at least (a) granulation of the active principle with a wetting liquid, (b) an extrusion step and (c) a Spheronisation, characterized in that the extrusion step (b) is carried out at low pressure.

In a preferred embodiment of the process the extrusion step is done with an extruder, preferably with an extruder of less pressure than a screw feed extruder, more preferably with a basket extruder.

In a preferred embodiment of the process there is at least one additional drying step, at least one additional coating step and/or at least one additional layering step.

A last main aspect of the invention is a pharmaceutical composition (called hereafter pharmaceutical composition C) produced by the process described above.

The examples in the following section are merely illustrative and the invention cannot be considered in any way as being restricted to these examples.

### EXAMPLES

### Example 1

Example of an Active Principle

(±)-2-((1-methyl-1H-pyrazol-5-yl)(thiophen-2-yl)methoxy)-N,N-dimethylethanamine or (±)-2-((1-methyl-1H-pyrazol-5-yl)(thiophen-2-yl)methoxy)-N,N-dimethylethanamine citrate of the formula 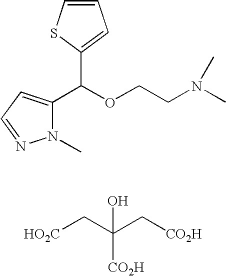
is already as base or citrate described in U.S. Pat. No. 6,410,582 and EP 1 072 266 including its synthesis and analgesic properties. This compound—also called active principle below—is used micronized.

### Example 2

General Process for Obtaining Coated Pellets:

In general the process for obtaining coated pellets involves five steps:
1. Granulation: preparation of a wet mass containing the active principle by blending and granulating the powder/s with a liquid, preferably water (10-30%). The preferred liquid (defined as “wetting liqid”) is water or alcohol/water mixtures e.g ethanol/water. A release controlling agent may be added here.
2. Extrusion: shaping the wet mass into cylinders especially into long rods or extrudates. In principle this can be and usually is done with high pressure, usually with a screw feed extruder. It can also be done with low pressure, e.g. with a basket extruder. In a screw feed extruder the wet mass is fed into the void of the screw which forces it up to, and through, the extruding orifice (“die”). The screws compact and densify the wet mass prior to extrusion and provide the force for extrusion. In a basket extruder the material is pushed through a mesh by rotating extrusion blades. With the sueve types in particular there is little precompaction prior to extrusion which generally results in extrudates of low density. Basket extruders are gravity-fed versions of the radial screw types.
3. Spheronisation: breaking up the extrudate and rounding off the particles into spheres. During this step some disintegrating agent may be added to avoid sticking.
4. Drying: drying of the pellets e.g. in a fluid bed at between 40° and 60° C. and for 20 minutes up to 1 hour.
5. Coating: The pellets will be coated with a coating containing a release controlling agent.

### Example 3

Example of a Pellet Core Using Manufacturing Process a According to example 1:

Table 1 shows the components of pellet core, the amount of every component for 100 g of formulation and the composition (%) for manufacturing process A:

TABLE 1
Composition of the pellet core
Composition Pellet Core. Manufacturing process A

| | | Theoretical | |
|---|---|---|---|
| | | amount 100 g | |
| Components | Composition % | formula | |
| 2-((1-methyl-1H- | 87.4 | 87.4 | |
| pyrazol-5- | | | |
| yl)(thiophen-2- | | | |
| yl)methoxy)-N,N- | | | |
| dimethylethanamine | | | |
| citrate | | | |
| Macrogol 8000 | 2.9 | 2.9 | |
| Microcrystalline | 9.7 | 9.7 | |
| +UZ,24/28 cellulose | | | |
| Total | 100.0% | 100.0 | g |

When granulating 10% to 30% of water (preferably 10%) was added to the powders.

### Example 4

1st Example of a Coated Pellet on a Core According to Example 3:

The pellet core according to example 3 will be coated (see step 5 in example 2) with a certain amount of ethylcellulose aqueous dispersion 25% dry, which is an excipient specifically for modified release and independent of the pH medium. The amounts coated are shown in Table 2.

When Spheronizing (Step 3 according to example 2 and before coating—step 5) 2% to 5% (preferably 5%) Microcrystalline cellulose was added to avoid sticking. Due to the fact that it is not possible to measure the amount sticking to the core and the amount remaining in the machine, the calculations and amounts in the table include ranges.

TABLE 2
Composition coated pellet 4.6-6.6%
Final compostion coated pellet

| Components | Composition % |
|---|---|
| 2-((1-methy1-1H- | 77.8-79.4 |
| pyrazol-5- | |
| yl)(thiophen-2- | |
| yl)methoxy)-N,N- | |
| dimethylethanamine | |
| citrate | |
| Macrogol 8000 | 2.6-2.7 |
| Microcrystalline | 13.1-13.4 |
| cellulose | |
| ethylcellulose | 6.6-4.6 |
| aqueous dispersion | |
| 25% dry | |
| (Surelease E-7-7050) | |
| Total | 100.0% |

### Example 5

2^{nd} Example of a Coated Pellet on a Core According to Example 3:

The pellet core according to example 3 will be coated (see step 5 in example 2) with a certain amount (different from example 4) of ethylcellulose aqueous dispersion 25% dry, which is an excipient specifically for modified release and independent of the pH medium. The amounts coated are shown in Table 3.

When Spheronizing (Step 3 according to example 2 and before coating—step 5) 2% to 5% (preferably 5%) Microcrystalline cellulose was added to avoid sticking. Due to the fact that it is not possible to measure the amount sticking to the core and the amount remaining in the machine, the calculations and amounts in the table include ranges.

TABLE 3
Final composition of coated pellet 7.9-9.9%
Final composition coated pellet

| Components | Composition % |
|---|---|
| 2-((1-methyl-1H- | 75.0-76.7 |
| pyrazol-5- | |
| yl)(thiophen-2- | |
| yl)methoxy)-N,N- | |
| dimethylethanamine | |
| citrate | |
| Macrogol 8000 | 2.5-2.6 |
| Microcrystalline | 12.6-12.9 |
| cellulose | |
| ethylcellulose | 9.9-7.9 |
| aqueous dispersion | |
| 25% dry (Surelease | |
| E-7-7050) | |
| Total | 100.0% |

### Example 6

3^{rd} Example of a Coated Pellet on a Core According to Example 3:

The pellet core according to example 3 will be coated (see step 5 in example 2) with a certain amount (different from examples 4 or 5) of ethylcellulose aqueous dispersion 25% dry (Surelease E-7-7050), which is an excipient specifically for modified release and independent of the pH medium. The amounts coated are shown in Table 4.

When Spheronizing (Step 3 according to example 2 and before coating—step 5) 2% to 5% (preferably 5%) Microcrystalline cellulose was added to avoid sticking. Due to the fact that it is not possible to measure the amount sticking to the core and the amount remaining in the machine, the calculations and amounts in the table include ranges.

TABLE 4
Final composition of coated pellet 7.7-9.5%
Final Composition coated pellet

| Components | Composition % |
|---|---|
| 2-((1-methyl-1H- | 75.3-76.9 |
| pyrazol-5- | |
| yl)(thiophen-2- | |
| yl)methoxy)-N,N- | |
| dimethylethanamine | |
| citrate | |
| Macrogol 8000 | 2.5-2.6 |
| Microcrystalline | 12.7-12.9 |
| cellulose | |
| ethylcellulose | 9.5-7.7 |
| aqueous dispersion | |
| 25% dry (Surelease | |
| E-7-7050) | |
| Total | 100.0% |

### Example 7

4^{th} Example of a Coated Pellet on a Core According to Example 3:

The pellet core according to example 3 will be coated (see step 5 in example 2) with a certain amount (different from examples 4, 5 or 6) of ethylcellulose aqueous dispersion 25% dry, which is an excipient specifically for modified release and independent of the pH medium. The amounts coated are shown in Table 5.

When Spheronizing (Step 3 according to example 2 and before coating—step 5) 2% to 5% (preferably 5%) Microcrystalline cellulose was added to avoid sticking. Due to the fact that it is not possible to measure the amount sticking to the core and the amount remaining in the machine, the calculations and amounts in the table include ranges.

TABLE 5
Final composition of coated pellet 4.3-6.2%
Final composition coated pellet

| Components | Composition % |
|---|---|
| 2-((1-methyl-1H- | 78-80 |
| pyrazol-5- | |
| yl)(thiophen-2- | |
| yl)methoxy)-N,N- | |
| dimethylethanamine | |
| citrate | |
| Macrogol 8000 | 2.6-2.7 |
| Microcrystalline | 13.1-13.3 |
| cellulose | |
| ethylcellulose | 4.3-6.2 |
| aqueous dispersion | |
| 25% dry (Surelease | |
| E-7-7050) | |
| Total | 100.0% |

### Example 8

Example of a Pellet Core Using Manufacturing Process B:

The table 6 shows the components of pellet core, the amount of every component for 100 g of formulation and the composition (%) for manufacturing process B:

TABLE 6
Composition from the core pellet.
Composition Pellet Core.
Manufacturing process B

| Components | Composition % |
|---|---|
| 2-((1-methyl-1H- | 75.2 |
| pyrazol-5- | |
| yl)(thiophen-2- | |
| yl)methoxy)-N,N- | |
| dimethylethanamine | |
| citrate | |
| Macrogol 8000 | 2.5 |
| Microcrystalline | 8.3 |
| cellulose | |
| ethylcellulose | 13.9 |
| aqueous dispersion | |
| 25% dry (Surelease | |
| E-7-7050) | |
| Total | 100.0% |

When granulating 10% to 30% of water (preferably 10%) was added to the powders.

The pellet core has been modified, adding ethylcellulose aqueous dispersion 25% dry. It enables the drug release control from the pellet core and makes the function of the layer easy which in the manufacturing process A (examples 4 to 7), is the main factor for controlling the drug release.

### Example 9

1^{st} Example of a Coated Pellet on a Core According to Example 8:

The pellet core according to example 8 will be coated with a certain amount of ethylcellulose aqueous dispersion 25% dry, which is an excipient specifically for modified release and independent of the pH medium. The amounts coated are shown in Table 7.

When Spheronizing (Step 3 according to example 2 and before coating—step 5) 2% to 5% (preferably 5%) Microcrystalline cellulose was added to avoid sticking. Due to the fact that it is not possible to measure the amount sticking to the core and the amount remaining in the machine, the calculations and amounts in the table include ranges.

TABLE 7
Composition coated pellet 12.8-14.8%
Final composition coated pellet %

| Components | Composition % |
|---|---|
| 2-((1-methyl-1H- | 61.1-62.5 |
| pyrazol-5- | |
| yl)(thiophen-2- | |
| yl)methoxy)-N,N- | |
| dimethylethanamine | |
| citrate | |
| Macrogol 8000 | 2.0-2.1 |
| Microcrystalline | 10.8-11.1 |
| cellulose | |
| ethylcellulose | 11.3-11.6 |
| aqueous dispersion | |
| 25% dry | |
| (Surelease E-7- | |
| 7050) (Intracore) | |
| ethylcellulose | 14.8-12.8 |
| aqueous dispersion | |
| 25% dry | |
| (Surelease E-7- | |
| 7050) (Layer) | |
| Total | 100.0% |

### Example 10

2^{nd} Example of a Coated Pellet on a Core According to Example 8:

The pellet core according to example 8 will be coated with a certain amount of ethylcellulose aqueous dispersion 25% dry ((Surelease E-7-7050)*, which is an excipient specifically for modified release and independent of the pH medium. The amounts coated are shown in Table 8.

When Spheronizing (Step 3 according to example 2 and before coating—step 5) 2% to 5% (preferably 5%) Microcrystalline cellulose was added to avoid sticking. Due to the fact that it is not possible to measure the amount sticking to the core and the amount remaining in the machine, the calculations and amounts in the table include ranges.

TABLE 8
Composition coated pellet 12.6-14.4%

| | Example 10 |
|---|---|
| | Final |
| Components | composition % |
| 2-((1-methyl-1H- | 63.0-61.0 |
| pyrazol-5- | |
| yl)thiophen-2- | |
| yl)methoxy)-N,N- | |
| dimethylethanamine | |
| citrate | |
| Macrogol 8000 | 2.0-2.1 |
| Microcrystalline | 10.8-11.1 |
| cellulose | |
| Ethylcellulose | 12.6-14.4 |
| aqueous dispersion | |
| 25% dry (Surelease | |
| E-7-7050) | |
| (Intracore) | |
| Ethylcellulose | 9.5-7.7 |
| aqueous dispersion | |
| 25% dry (Surelease | |
| E-7-7050) (Layer) | |
| Total | 100.0 |

### Example 11

Different Approaches for the Extrusion Step (Step 2 of Example 2):

Two different approaches were tried to execute the extrusion step (Step 2 of example 2). The usual way of extruding using high pressure surprisingly was not working at all so that there was the risk that no extrusion would be possible. Still, fortunately the extrusion at low pressure using e.g a basket extruder was working with very comfortable results.

The results of two parallel example are shown in Table 9:

| | | Extruder | | | |
|---|---|---|---|---|---|
| | Compo- | | parameters | | |
| | sition | | Screen | | |
| | (w/w) | | size | Speed | |
| Formulations | (g) | Type | (mm) | (rpm) | Observations |
| 2-((1- | 100/20 | High | 0.5 | 70 | The machine |
| methyl- | | Pressure, | | | was overload. |
| 1H- | | Vertikal* | | | After a few |
| pyrazol-5- | | extruder | | | seconds, the |
| yl)(thiophen- | | | | | product started |
| 2-yl)methoxy)- | | | | | to compact |
| N,N- | | | | | before the |
| dimethyl | | | | | screen, which |
| ethanamine | | | | | actually |
| Citrate/ | | | | | blocked. |
| Water | | | | | Deflection of |
| | | | | | the screen was |
| | | | | | noticed. When |
| | | | | | opening the |
| | | | | | machine, |
| | | | | | densified |
| | | | | | material was |
| | | | | | found at the |
| | | | | | screen and on |
| | | | | | the screw. |
| 2-((1- | 100/20 | Low | 0.5 | — | The extrusion |
| methyl- | | pressure, | | | of the product |
| 1H- | | basket** | | | was good. No |
| pyrazol-5- | | extruder | | | secondary |
| yl)(thiophen-2- | | | | | agglomeration |
| yl)methoxy)- | | | | | of the |
| N,N- | | | | | extrudates. |
| dimethyl | | | | | |
| ethanamine | | | | | |
| Citrate/ | | | | | |
| Water | | | | | |

*DG-L1: Lab. Dome Extruder (a Screw feed extruder)
**KAR-75: Lab. Basket Extruder

### Example 12

Example of a Complete Production According to Manufacturing Process A

1) 2-((1-methyl-1H-pyrazol-5-yl)(thiophen-2-yl)methoxy)-N,N-dimethylethanamine citrate and microcrystalline cellulose are loaded into a mixer-granulator and mixed.
2) Macrogol 8000 is dissolved with purified water in a stainless steel bowl. The purified water will be evaporated during the dried process in the fluid bed (step 6).
3) The solution prepared in the second step is added slowly on the mixture contained in the granulator. The mass is kneaded until the granulate is obtained.
4) The granulate is extruded in a basket extruder.
5) The extrudates are transformed to spherical pellets in the spheronization process.
6) The pellets obtained are dried in a fluid bed for 0.5 h above 40° C.
7) The core pellets obtained are sieved to get a more narrow size distribution.
8) The pellets are coated with ethylcellulose aqueous dispersion.
9) Finally the pellets coated are sieved.

### Example 13

Example of a Complete Production According to Manufacturing Process B

1) 2-((1-methyl-1H-pyrazol-5-yl)(thiophen-2-yl)methoxy)-N,N-dimethylethanamine citrate, Micocrystalline cellulose and macrogol 8000 are loaded into a mixer-granulator and mixed.
2) Ethylcellulose aqueous dispersion is sprayed to the mass until to obtain a uniform granulate.
3) The granulate is dried in a fluid bed for 0.5 h above 40° C.
4) The dried granulate is loaded into a mixer-granulator and mixed.
5) Eethylcellulose aqueous dispersion is sprayed to the mass until to obtain a uniform granulate.
6) The granulate is extruded in a basket extruder.
7) The extrudates are transformed to spherical pellets in the spheronization process.
8) The pellets obtained are dried in a fluid bed for 0.5 h above 40° C.
9) The core pellets obtained are sieved to get a more narrow size distribution.
10) The pellets are coated with ethylcellulose aqueous dispersion.
11) Finally the pellets are sieved.

What is claimed is:

1. Pharmaceutical composition for the oral administration of a pirazole compound of formula (XI) 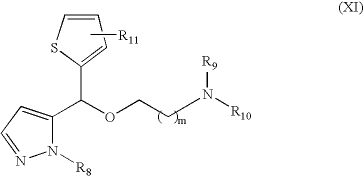
with
m is 1 or 2;
R₈ is selected from hydrogen, fluoride, chloride, bromide and C₁₋₄-Alkyl, especially methyl;
R₉ and R₁₀ are independently selected from C₍₁₋₄₎-Alkyl or together with the Nitrogen form an azaheterocyclic ring;
R₁₁ is selected from the group consisting of hydrogen, fluoride, chloride, bromide, C₁₋₄-Alkyl, especially methyl or trifluoromethyl, and O—C₁₋₄-Alkyl, especially methoxy,
of one of its enantiomers or a mixture of its enantiomers in any mixing ratio including the racemate,
including pharmaceutically acceptable salts or solvates, (collectively called active principle hereafter)
comprising:
≧70% by weight of the active principle
in at least one of its distinct parts.

2. Pharmaceutical composition for the oral administration of a pirazole compound of formula (X) according to claim 1 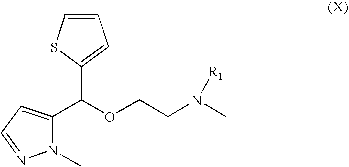
with R₁ being H or CH₃,
of one of its enantiomers or a mixture of its enantiomers in any mixing ratio including the racemate,
including pharmaceutically acceptable salts or solvates, (collectively called active principle hereafter)
comprising:
≧70% by weight of the active principle
in at least one of its distinct parts.

3. Pharmaceutical composition for the oral administration of 2-((1-methyl-1H-pyrazol-5-yl) (thiophen-2-yl)methoxy)-N,N-dimethylethanamine of formula I according to claim 1 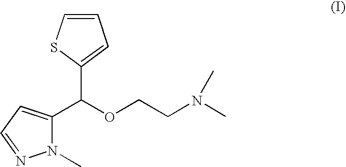
of one of its enantiomers or a mixture of its enantiomers in any mixing ratio including the racemate,
including pharmaceutically acceptable salts or solvates,
(collectively called active principle hereafter)
comprising:
≧70% by weight of the active principle
in at least one of its distinct parts.

4. Pharmaceutical composition according to claim 1 for the oral administration of (+)-2-((1-methyl-1H-pyrazol-5-yl)(thiophen-2-yl)methoxy)-N,N-dimethylethanamine or (−)-2-((1-methyl-1H-pyrazol-5-yl)(thiophen-2-yl)methoxy)-N,N-dimethylethanamine according to formula II or III respectively or any mixture of the compounds of formula II or III including a racemic mixture (collectively called active principle hereafter) 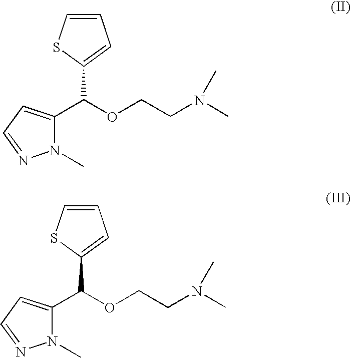
including pharmaceutically acceptable salts or solvates,
comprising:
≧70% by weight of the active principle
in at least one of its distinct parts.

5. Pharmaceutical composition for the oral administration of 2-((1-methyl-1H-pyrazol-5-yl) (thiophen-2-yl)methoxy)-N-methylethanamine of formula IV according to claim 1 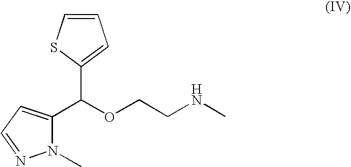
of one of its enantiomers or a mixture of its enantiomers in any mixing ratio including the racemate,
including pharmaceutically acceptable salts or solvates,
(collectively called active principle hereafter)
comprising:
≧70% by weight of the active principle
in at least one of its distinct parts.

6. Pharmaceutical composition according to claim 1 for the oral administration of (+)-2-((1-methyl-1H-pyrazol-5-yl)(thiophen-2-yl)methoxy)-N-methylethanamine or (−)-2-((1-methyl-1H-pyrazol-5-yl)(thiophen-2-yl)methoxy)-N-methylethanamine according to formula V or VI respectively or any mixture of the compounds of formula V or VI including a racemic mixture (collectively called active principle hereafter) 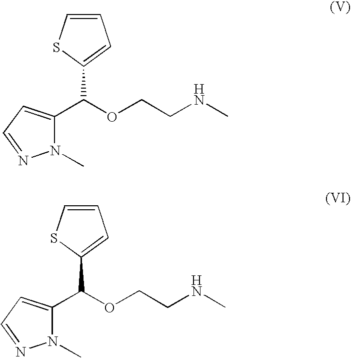
including pharmaceutically acceptable salts or solvates,
comprising:
≧70% by weight of the active principle
in at least one of its distinct parts.

7. Pharmaceutical Composition according to claim 1, wherein the amount of active principle is ≧500 mg and ≦1100 mg, preferably ≧600 mg and ≦1000 mg, more preferably ≧700 mg and ≦900 mg, most preferably 800 mg.

8. Pharmaceutical Composition according to claim 1, wherein the amount of the free base of the active principle is ≧250 mg and ≦650 mg, preferably ≧350 mg and ≦600 mg, more preferably ≧400 mg and ≦500 mg, most preferably 450 mg.

9. Pharmaceutical Composition according to claim 1, wherein the amount of active principle is ≧100 mg and ≦500 mg, preferably ≧150 mg and ≦450 mg, more preferably ≧200 mg and ≦400 mg.

10. Pharmaceutical Composition according to claim 1, wherein the amount of the free base of the active principle is ≧50 mg and ≦300 mg, preferably ≧85 mg and ≦250 mg, more preferably ≧120 mg and ≦200 mg.

11. Pharmaceutical Composition according to any of claim 1, wherein the distinct part is coated with a coating agent, preferably a release control coating agent, more preferably a cellulose derivative, most preferably ethyl cellulose.

12. Pharmaceutical Composition according to claim 1, wherein the distinct part comprises an extrusion/spheronisation enhancer, preferably a cellulose derivative.

13. Pharmaceutical Composition according to claim 1, wherein the distinct part comprises a plasticizer, preferably a polyethylene glycol, more preferably a macrogol.

14. Pharmaceutical Composition according to claim 1, wherein the distinct part comprises a coating agent, preferably a release control coating agent, more preferably a cellulose derivative, most preferably ethyl cellulose.

15. Pharmaceutical Composition according to claim 1, wherein the Pharmaceutical Composition is a tablet, a capsule, a pellet, a sachet or a suspension.

16. Pharmaceutical Composition according to claim 1, wherein the distinct part is the core of a tablet, a layer of a tablet, the nucleus of a pellet or a layer of a pellet, preferably the nucleus of a pellet.

17. Pharmaceutical Composition according to claim 1,
comprising:
≧75% by weight of the active principle, preferably ≧75% to 95%, more preferably 80% to 95%,
in at least one of its distinct parts.

18. Pharmaceutical Composition according to claim 1 overall comprising:
≧60% by weight of the active principle, preferably ≧70%, preferably 70% to 95%, most preferably 75% to 95%, preferably 80% to 95%.

19. Pharmaceutical Composition according to claim 1 wherein the active principle is 2-((1-methyl-1H-pyrazol-5-yl)(thiophen-2-yl)methoxy)-N,N-dimethylethanamine citrate:

20. Pharmaceutical composition for the oral administration of a pirazole compound of formula (XI) 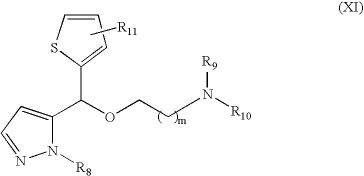
with
m is 1 or 2;
R₈ is selected from hydrogen, fluoride, chloride, bromide and C₁₋₄-Alkyl, especially methyl;
R₉ and R₁₀ are independently selected from C₍₁₋₄₎-Alkyl or together with the Nitrogen form an azaheterocyclic ring;
R₁₁ is selected from the group consisting of hydrogen, fluoride, chloride, bromide, C₁₋₄-Alkyl, especially methyl or trifluoromethyl, and O—C₁₋₄-Alkyl, especially methoxy,
of one of its enantiomers or a mixture of its enantiomers in any mixing ratio including the racemate,
including pharmaceutically acceptable salts or solvates, (collectively called active principle hereafter)
comprising:
(a) a core comprising the active principle and a Extrusion/Spheronisation enhancer,
(b) a coating comprising a coating agent.

21-43. (canceled)
